(19) United States
(12) Patent Application Publication
(10) Pub. No.: US 2013/0261391 A1
(43) Pub. Date: Oct. 3, 2013

(54) PROPULSION ASSEMBLY FOR ENDOSCOPE

(71) Applicant: FUJIFILM CORPORATION, Tokyo (JP)

(72) Inventors: Takumi DEJIMA, Ashigarakami-gun (JP); Tsuyoshi ASHIDA, Ashigarakami-gun (JP); Takayuki NAKAMURA, Ashigarakami-gun (JP); Nobuyuki TORISAWA, Ashigarakami-gun (JP); Naoyuki MORITA, Ashigarakami-gun (JP)

(73) Assignee: FUJIFILM Corporation, Tokyo (JP)

(21) Appl. No.: 13/853,118

(22) Filed: Mar. 29, 2013

(30) Foreign Application Priority Data
Mar. 30, 2012 (JP) 2012-078979

(51) Int. Cl. A61B 1/00

(52) U.S. Cl. CPC A61B 1/00156
USPC 600/114

(57) ABSTRACT

A propulsion assembly for mounting on a tip device of an endoscope includes a barrel sleeve having inner and outer surfaces. A flexible endless track device is disposed to extend along the inner and outer surfaces of the barrel sleeve, for endlessly moving in an axial direction, for propulsion by contacting a wall of a body cavity. A drive gear is engaged with the endless track device, for moving the same. A driving mechanism rotates the drive gear. Engaging teeth are formed on the endless track device at a predetermined pitch, arranged serially, moved by the drive gear in mesh therewith. A peripheral surface of the engaging teeth is curved without a sharp form. Furthermore, a support sleeve is disposed between the driving mechanism and the endless track device, for supporting the drive gear in a rotatable manner.

### BACKGROUND OF THE INVENTION

1. Field of the Invention

The present invention relates to a propulsion assembly for an endoscope. More particularly, the present invention relates to a propulsion assembly for an endoscope in which a wall of a body cavity can be kept safe without mechanical influence during propulsion of the endoscope.

2. Description Related to the Prior Art

An endoscope is an instrument in the medical field for diagnosis and treatment. The endoscope has an elongated tube and a tip device at a distal end. A CCD or the like is incorporated in the tip device and entered in a body cavity of a patient's body. An image is obtained by the CCD, displayed on a display panel, and viewed for diagnosing the body cavity.

A propulsion assembly for propelling an elongated tube of the endoscope has been suggested recently. U.S. Ser. No. 2005/272,976 (corresponding to JP-A 2005-253892) discloses the propulsion assembly including a support sleeve and an endless track device. The support sleeve is mounted on the tip device of the endoscope. The endless track device is endlessly movable around the support sleeve. An upper run (working run) of the endless track device contacts a wall of a gastrointestinal tract and is traveled, so that the endless track device moves the tip device of the endoscope in a distal direction through the gastrointestinal tract more deeply according to friction of the endless track device with the wall. It is effective in facilitating entry of the endoscope into the gastrointestinal tract of a tortuous form, such as a large intestine, even with low skill in the manipulation of the endoscope.

The above document discloses an endless track device disposed to extend between magnetic rollers in an endlessly movable manner. A motor rotates a wire device so as to rotate a magnetic bar at a distal end of the wire device to move the endless track device. The magnetic bar is in a form in which lines of N and S poles are wound helically, and operates as a worm gear. The magnetic rollers have N and S poles on its outer surface and operate as a worm wheel . If propulsion of the endless track device is stopped by increasing resistance of the contact with a wall of the body cavity, rotation of the magnetic bar is stopped. Overload occurs to the wire device with magnets and also to the motor for the wire device, so that the wire device and the motor may be broken. Even when the motor is not broken, force of the endless track device is applied to the wall of the body cavity, which may be scratched or damaged seriously.

It is conceivable to form engaging teeth on the outer surface of the endless track device, and to mesh a drive gear with the engaging teeth of the endless track device for endlessly moving the endless track device. However, there is a problem in low safety, as the engaging teeth is opposed to and contacts a wall of the body cavity on the outside of the endless track device.

### SUMMARY OF THE INVENTION

In view of the foregoing problems, an object of the present invention is to provide a propulsion assembly for an endoscope in which a wall of a body cavity can be kept safe without mechanical influence during propulsion of the endoscope.

In order to achieve the above and other objects and advantages of this invention, a propulsion assembly for mounting on a tip device of an endoscope includes a barrel sleeve having inner and outer surfaces. An endless track device has flexibility, is disposed to extend around the inner and outer surfaces of the barrel sleeve, for endlessly moving in an axial direction of the tip device, for propulsion by contacting a wall of a body cavity. A drive gear is engaged with the endless track device, for moving the endless track device. A drive sleeve rotates the drive gear. Engaging teeth are formed on the endless track device at a predetermined pitch, arranged serially, moved by the drive gear in mesh therewith, wherein a peripheral surface of the engaging teeth is curved without a sharp form.

The peripheral surface of the engaging teeth is arcuate according to a semi-circular shape.

In another preferred embodiment, the peripheral surface of the engaging teeth is in a quadrilateral shape as viewed in a section, and corners of the quadrilateral shape are curved arcuately.

The drive gear is helical and has gear teeth inclined with respect to the axial direction. The engaging teeth are inclined with respect to the axial direction and meshed with the gear teeth.

Furthermore, a support sleeve is disposed between the drive sleeve and the barrel sleeve, for supporting the drive gear in a rotatable manner.

The endless track device has an annular surface, and covers the barrel sleeve in a bag shape.

In still another preferred embodiment, the endless track device includes a plurality of endless belts disposed to extend in the axial direction and arranged in a circumferential direction of the barrel sleeve.

The endless track device includes a lower run disposed to extend along the inner surface of the barrel sleeve, wherein the engaging teeth on the lower run are meshed with the drive gear. An upper run is disposed to extend along the outer surface of the barrel sleeve, for moving in a direction reverse to the lower run in contact with the wall of the body cavity.

Furthermore, at least one recess is formed in an outer surface of the support sleeve. An inclined surface is formed at least at an end of the recess on a distal side with respect to the axial direction, for smoothing movement of the lower run relative to the barrel sleeve.

Furthermore, plural rollers are supported on the barrel sleeve in a rotatable manner, for keeping the lower run movable in cooperation with the drive gear.

Also, the endless track device includes first and second edge portions, disposed at respectively ends of an initially prepared sleeve material or strip material, for defining an endless form by attachment to one another. The second edge portion is disposed on a proximal side from the first edge portion in the axial direction when positioned in the lower run. A joint structure attaches the first and second edge portions to one another by positioning the first edge portion between the barrel sleeve and the second edge portion in an overlaid manner.

In another preferred embodiment, the endless track device includes first and second edge portions, disposed at respectively ends of an initially prepared sleeve material or strip material, for defining an endless form by attachment to one another. The second edge portion is disposed on a proximal side from the first edge portion in the axial direction when positioned in the lower run. A joint structure attaches the first and second edge portions to one another by positioning the second edge portion between the barrel sleeve and the first edge portion in an overlaid manner.

Therefore, a wall of a body cavity can be kept safe without mechanical influence during propulsion of the endoscope, because the engaging teeth have a curved shape.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope and a propulsion assembly in combination;

FIG. 2 is a perspective view illustrating the propulsion assembly of which an endless track device is developed;

FIG. 3 is an exploded perspective view illustrating the propulsion assembly;

FIG. 4 is an exploded perspective view illustrating a drive sleeve, torque wire devices and motors;

FIG. 5 is a perspective illustrating portions of the endless track device before attachment;

FIG. 6 is a perspective illustrating the portions of the endless track device after attachment and drive gears;

FIG. 7 is a vertical section illustrating the propulsion assembly;

FIG. 8 is an explanatory view in a cross section illustrating the endless track device;

FIG. 9 is a cross section illustrating the propulsion assembly;

FIG. 10 is a vertical section illustrating portions of the propulsion assembly with the drive gears;

FIG. 11 is a vertical section illustrating another preferred endless track device in which engaging teeth with an arcuate shape are formed on an outer surface;

FIG. 12 is a vertical section illustrating one preferred endless track device of which portions of the endless track devices are attached in a reverse manner;

FIG. 13 is a vertical section illustrating still another preferred structure of coupling gears of which both ends are supported.

### DETAILED DESCRIPTION OF THE PREFERRED EMBODIMENT(S) OF THE PRESENT INVENTION

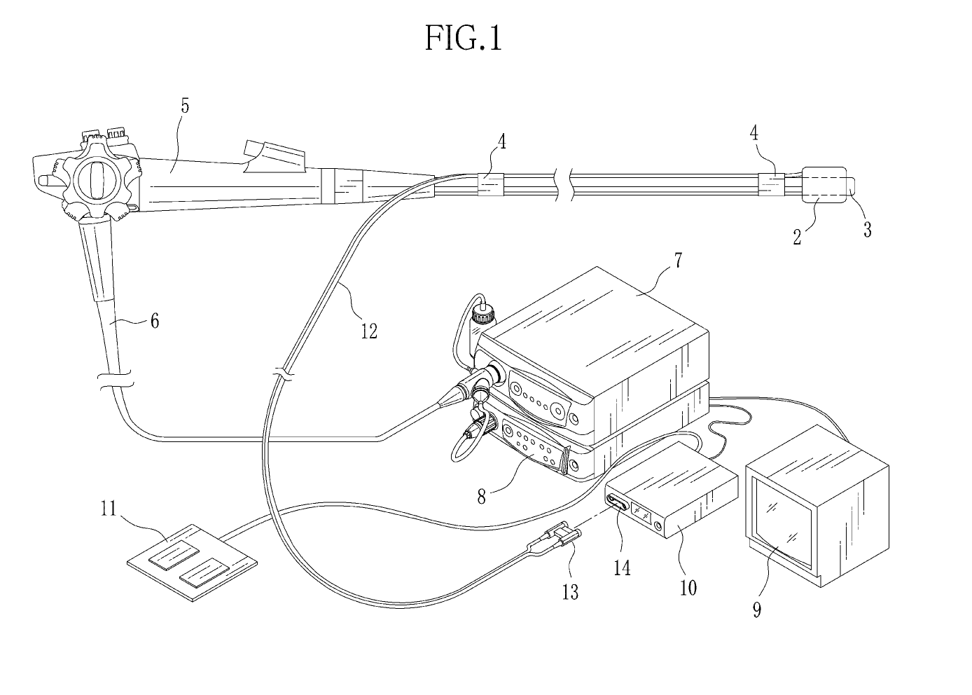

In FIG. 1, a propulsion assembly 2 is for use with an endoscope. The propulsion assembly 2 is fitted around a tip device 3 of the endoscope. The endoscope includes an image sensor, lighting windows, a steering device, an elongated tube, a handle 5, steering wheels and the like. The image sensor is incorporated in the tip device 3, and is a CCD or CMOS image sensor. The lighting windows are formed in the tip device 3 and emit light. The image sensor images an object in a body cavity illuminated with the light from the lighting windows, such an object as a wall of a stomach or intestine of a gastrointestinal tract of a patient. The steering device is disposed at a proximal end of the tip device 3 for steering to enter the tip device 3 in the body cavity to reach the object. The propulsion assembly 2 operates to facilitate the entry of the tip device 3. The steering wheels are disposed on the handle 5, and manually rotated to operate the steering device for bending up and down and to the right and left .

The handle 5 includes a button and an end sleeve. The button is operable to change over the supply and suction of air or water. The end sleeve has an instrument opening where a biopsy forceps or other medical device is advanced. A universal cable 6 extends from the handle 5, and is connected to a light source apparatus 7 and a processing apparatus 8. Light from a lamp in the light source apparatus 7 is guided by a light guide fiber extending through the universal cable 6 and the endoscope to the lighting windows. The processing apparatus 8 processes an image signal from the universal cable 6 in the signal processing suitably. A display panel 9 is driven to display the image of the image signal. The processing apparatus 8 discerns the type information of the endoscope for use according to the input information from the endoscope through the universal cable 6. The processing apparatus 8 automatically changes over the control and/or display suitably according to the type information, typically if the control with differences for the types is required in the course of the manipulation, or if the display with differences for the types is required on the display panel 9.

An actuating apparatus 10 or controller is connected with the processing apparatus 8 electrically. The actuating apparatus 10 actuates and controls the propulsion assembly 2. A foot switch 11 is connected with the actuating apparatus 10 for starting the propulsion assembly 2 to move endlessly. A wire sheath 12 of a dual lumen form extends from a proximal end of the propulsion assembly 2. An adhesive tape 4 or surgical tape positions the wire sheath 12 on the elongated tube of the endoscope at suitable points. The wire sheath 12 extends properly into the body cavity even upon moving the endoscope into the body cavity or during the manipulation.

First and second torque wire devices 30a and 30b are disposed to extend discretely through the wire sheath 12. Distal end portions of the wire devices 30a and 30b are coupled to a driving mechanism (sleeve) of the propulsion assembly 2. The wire devices 30a and 30b are flexible but have high torsional rigidity so that torque applied to their proximal end is transmitted by those to their distal end substantially without attenuation. A key coupling device 13 for plug-in is disposed at the proximal end of the wire devices 30a and 30b. A rotating coupling 14 for plug-in is disposed in the actuating apparatus 10, and connected mechanically with the key coupling device 13. First and second motors are incorporated in the actuating apparatus 10. When the key coupling device 13 is plugged to the rotating coupling 14, each of the wire devices 30a and 30b is ready to rotate with one of the first and second motors.

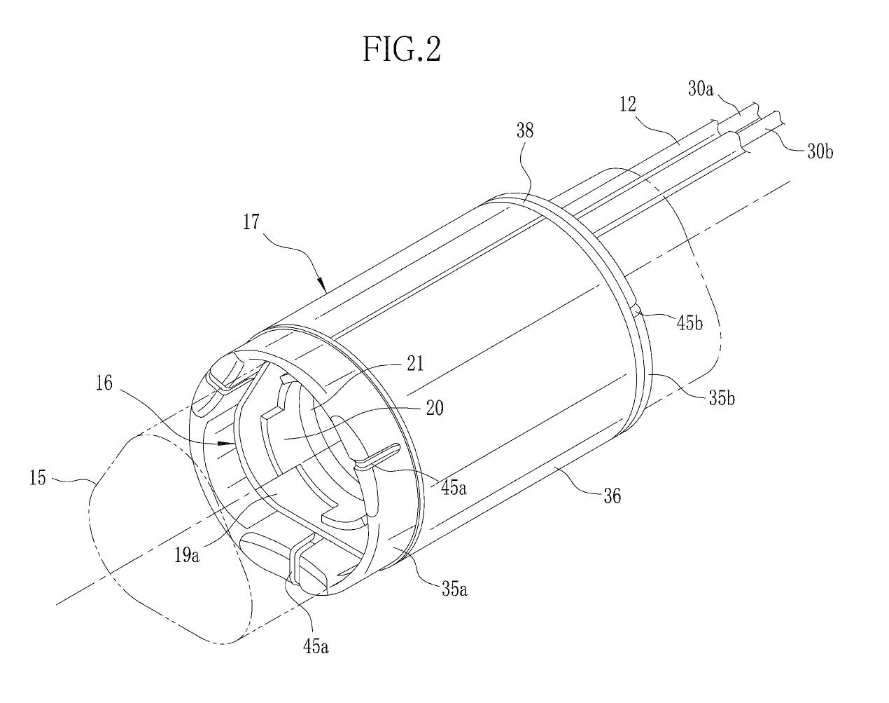

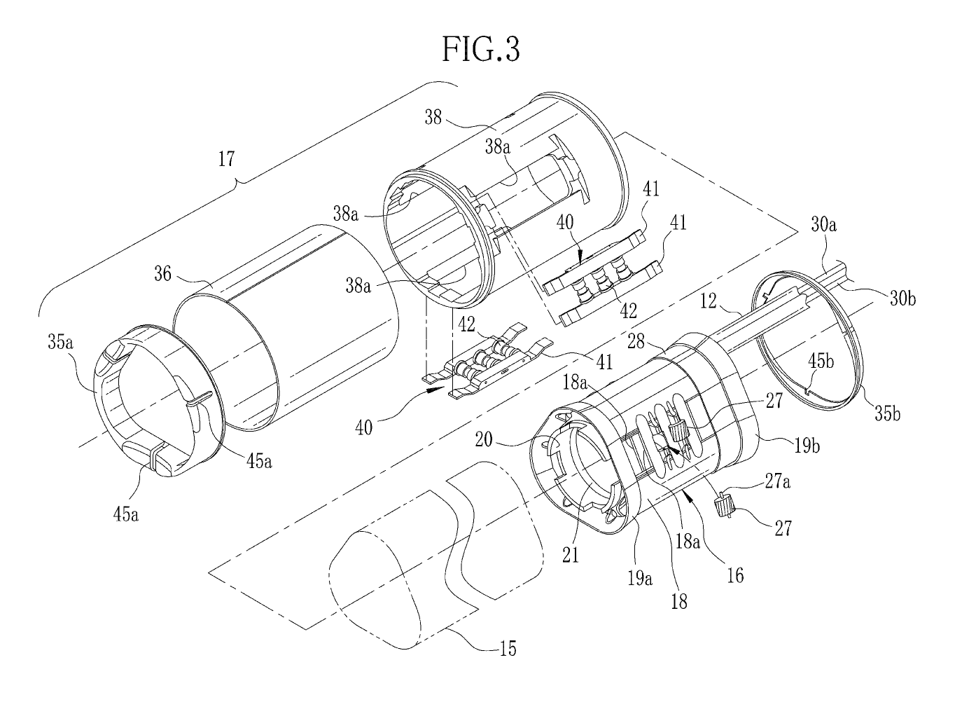

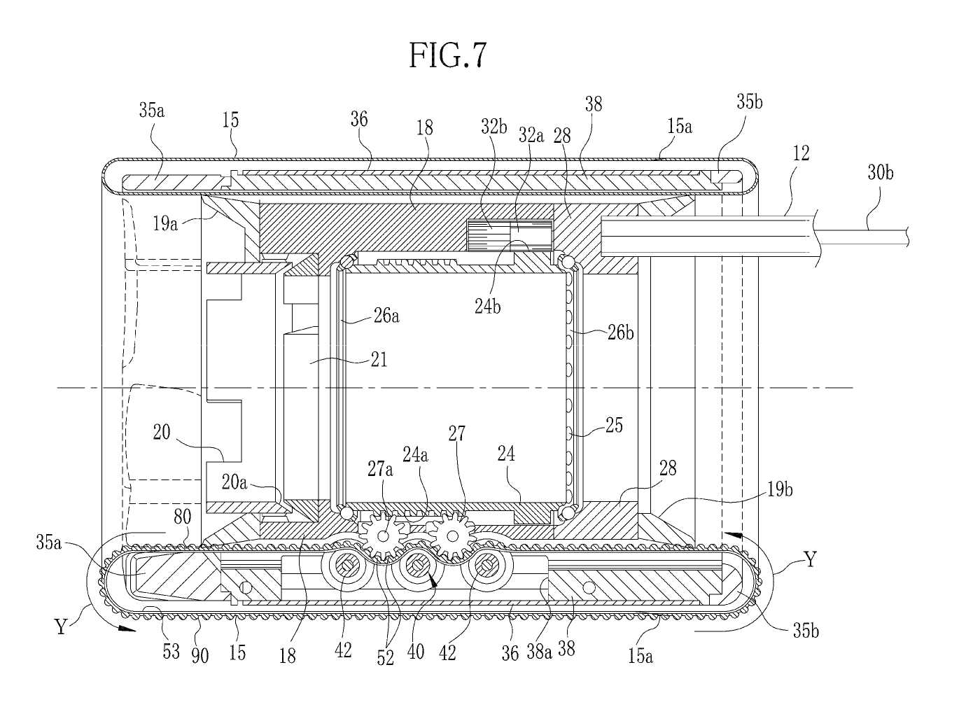

The propulsion assembly 2 is used effectively specially for colonoscopy, because of manipulation for advance and pull in the sigmoid colon or transverse colon. The propulsion assembly 2 is substantially cylindrical. An endless track device 15 or membrane or toroidal device is disposed on the outside of the propulsion assembly 2, is constituted by a flexible sheet of synthetic resin with sufficient rigidity. In FIGS. 2 and 3, the endless track device 15 is depicted in a developed form of a sleeve material for understanding. A final form of the endless track device 15 is in a ring shape or toroidal shape after connecting first and second edge portions of the initially prepared sleeve material. The endless track device 15 has an annular surface. See FIG. 7. In FIGS. 2-7, a distal side for protruding the tip device 3 is depicted on the left side. A proximal side near to the handle 5 of the endoscope is depicted on the right side.

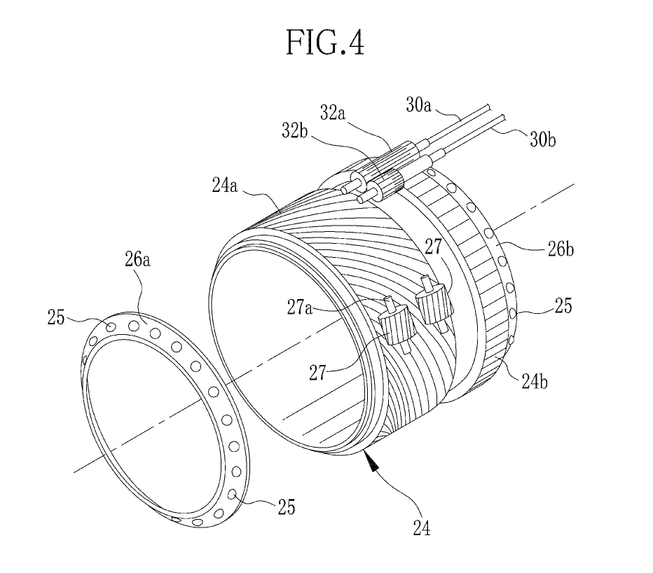

In FIGS. 2 and 3, the propulsion assembly 2 includes a drive unit 16 or inner unit, and a barrel unit 17 or outer unit. The drive unit 16 is disposed inside the endless track device 15. The barrel unit 17 is disposed around the drive unit 16. The drive unit 16 includes a support sleeve 18, a cap ring 28, a distal cover flange 19a for wiping, a proximal cover flange 19b for wiping, a clamping sleeve 20 or collet sleeve, a sealing device 21 (in a C-shape) or C-ring or collet head, and a drive sleeve 24 of FIG. 4. The support sleeve 18 has a cylindrical inner surface and an outer surface in a shape of a triangular prism. The cap ring 28 is in a triangular shape, and retained to a proximal end of the support sleeve 18 by a screw, press-fit or caulking. The cover flanges 19a and 19b are attached to respectively the distal end of the support sleeve 18 and the proximal end of the cap ring 28. The clamping sleeve 20 is helically engaged with a thread formed inside the support sleeve 18, and rotates to move in the axial direction. The sealing device 21 is formed from synthetic resin with resiliency, and has a diameter changeable by movement of the clamping sleeve 20 in the axial direction. The drive sleeve 24 is a driving mechanism supported inside the support sleeve 18 in a rotatable manner.

In FIG. 4, the propulsion assembly 2 includes bearing rings 26a and 26b, each of which is constituted by plural bearing balls 25 arranged annularly. The bearing rings 26a and 26b support ends of the drive sleeve 24 on an inner surface of the support sleeve 18 in a rotatable manner. The cap ring 28 is secured to a proximal end of the support sleeve 18, and prevents the drive sleeve 24 from dropping out . Worm gear teeth 24a or thread, and spur gear teeth 24b are arranged on an outer surface of the drive sleeve 24. Two rotatable drive gears 27, or worm wheels or roller wheels with helical gear teeth, are supported on the support sleeve 18, and meshed with the worm gear teeth 24a through openings in the support sleeve 18. Three pairs of the drive gears 27 are arranged equiangularly from one another around the drive sleeve 24. When the drive sleeve 24 rotates, the drive gears 27 rotate around a gear shaft 27a in the same direction simultaneously.

A distal end of the wire sheath 12 is attached to the inside of the proximal end of the cap ring 28 by use of adhesion or thermal welding. Distal ends of first and second torque wire devices 30a and 30b protruding from the wire sheath 12 extend to pass through holes in the cap ring 28. First and second coupling gears 32a and 32b or pinions are firmly connected with distal ends of the wire devices 30a and 30b. As illustrated in the drawing, rotational shafts protrude from respectively the coupling gears 32a and 32b as rotational centers. The shafts are received in holes formed in the support sleeve 18, to keep the coupling gears 32a and 32b rotatable. Only the first coupling gear 32a of the first wire device 30a is meshed with the spur gear teeth 24b of the drive sleeve 24. The second coupling gear 32b of the second wire device 30b is meshed with the first coupling gear 32a but not with the spur gear teeth 24b. Thus, the drive sleeve 24 is driven by rotation of the first coupling gear 32a in connection with the first wire device 30a. However, the wire devices 30a and 30b are driven by torques generated by respectively the motors. The second coupling gear 32b is rotated in a direction opposite to that of the first coupling gear 32a. The torque from the second wire device 30b is added to the torque of the first coupling gear 32a, so that the drive sleeve 24 can be rotated with a high torque.

Each of the cover flanges 19a and 19b includes a flange edge shaped to increase a width in the axial direction. The flange edge receives an inner surface of the endless track device 15 with closeness while the endless track device 15 turns around. The flange edge prevents various materials from pull into the propulsion assembly 2 together with the moving outer surface of the endless track device 15, the materials including foreign material and tissue of a body part.

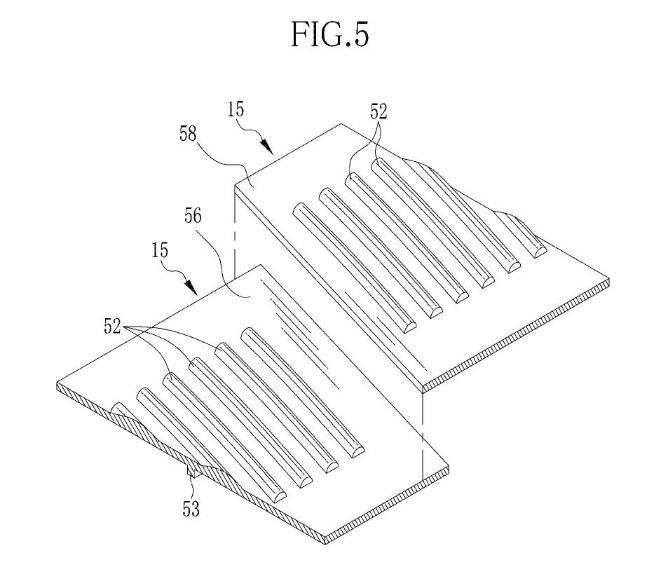

A distal end of the clamping sleeve 20 has a pattern of projections and recesses arranged in the circumferential direction. A special screw driving device for the clamping sleeve 20 is entered for engagement with the clamping sleeve 20 in the proximal direction. The clamping sleeve 20 is rotated in a predetermined direction by the screw driving device, and thus shifts in the proximal direction. A tapered end surface 20a of the clamping sleeve 20 in FIG. 5 presses the sealing device 21, which deforms to decrease the diameter. Accordingly, an inner surface of the sealing device 21 is strongly pressed on a peripheral surface of the tip device 3 for firmly fitting the support sleeve 18 thereon.

The barrel unit 17 includes a distal support ring 35a or bumper ring, a cover sheet 36 for shielding, a barrel sleeve 38 (outer sleeve) for supporting idler rollers, and a proximal support ring 35b or bumper ring, in a sequence in the proximal direction. The barrel unit 17 is combined with the drive unit 16 and the endless track device 15 according to the steps as follows.

In FIGS. 2 and 3, a sheet material for the endless track device 15 in a developed form is formed in a cylindrical shape. The drive unit 16 is positioned so that its outer surface is covered inside the cylindrical shape of the sheet material. The drive unit 16 with the endless track device 15 is entered in the barrel sleeve 38. Three holder openings 38a are formed in the barrel sleeve 38 to extend in the axial direction, and arranged equiangularly from one another with 120 degrees. Roller mechanisms 40 are mounted in respectively the holder openings 38a.

In FIGS. 2-7, the roller mechanisms 40 include three idler rollers 42, and a pair of roller supports 41 or frames for supporting the idler rollers 42 in alignment. The roller supports 41 are resilient thin plates of metal, and are fixed to the barrel sleeve 38 by fitting their ends in end portions of the holder openings 38a. A center of the roller supports 41 in the longitudinal direction becomes curved to enter an inner space in the barrel sleeve 38 through the holder openings 38a. The idler rollers 42 supported by the roller supports 41 press the endless track device 15 toward the drive gears 27 owing to the curved form of the roller supports 41. As a result, the endless track device 15 is tensioned tightly between the drive gears 27 and the idler rollers 42. See FIG. 7. Two of the drive gears 27 and three of the idler rollers 42 are alternate with one another to constitute one array for running the endless track device 15 with tension. Three of such arrays are arranged equiangularly around the axis of the drive and barrel units 16 and 17. There is degree of freedom in one of the idler rollers 42 disposed at the center in relation to the longitudinal direction of the roller supports 41, because the center roller is supported by the opening extending longitudinally. A relative position of the endless track device 15 to two lateral rollers included in the idler rollers 42 is automatically adjusted for supporting the endless track device 15 with the drive gears 27 in an optimally balanced manner.

The roller mechanisms 40 are fitted in the holder openings 38a fixedly on the barrel sleeve 38. The idler rollers 42 project to the inside of the barrel sleeve 38 and keep the barrel sleeve 38 immovable in the axial direction relative to the drive unit 16. The endless track device 15 is tensioned while the roller mechanisms 40 are combined with the barrel sleeve 38. The support rings 35a and 35b are fixed to respectively the distal and proximal ends of the barrel sleeve 38. Three guide grooves 45a are formed in the distal support ring 35a. Three guide grooves 45b are formed in the proximal support ring 35b. The guide grooves 45a and 45b are aligned with the roller mechanisms 40 in the axial direction.

The cover sheet 36 tightly covers the outer surface of the barrel sleeve 38 together with the roller mechanisms 40.

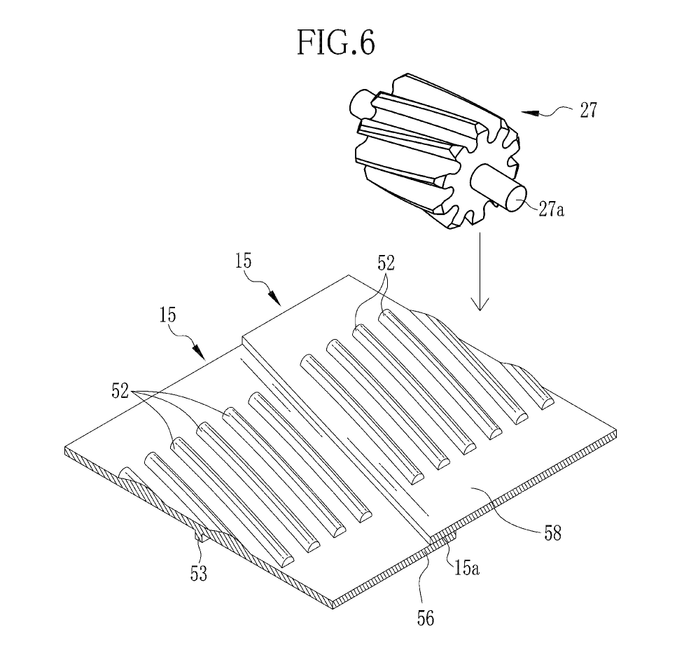

The sleeve material of the endless track device 15 in a developed form is positioned between the drive and barrel units 16 and 17. Those units are combined with one another, before first and second edge portions 56 and 58 of the sleeve material of the endless track device 15 are turned over and connected with one another. See FIGS. 5 and 6. A joint structure 15a of the endless track device 15 is formed. Note that inclinations can be preferably formed with the first and second edge portions 56 and 58 of the sleeve material of the endless track device 15, so that the joint structure 15a can have a small thickness without an excessive unevenness of the thickness. In FIG. 7, an assembled structure of the propulsion assembly 2 is schematically illustrated. The endless track device 15 can have an inner space to wrap the barrel unit 17 entirely in the toroidal shape. It is possible to fill the inner space with suitable fluid, such as air, physiological saline water, colloid of synthetic resin, oil, grease, lubricant fluid of various types, and the like.

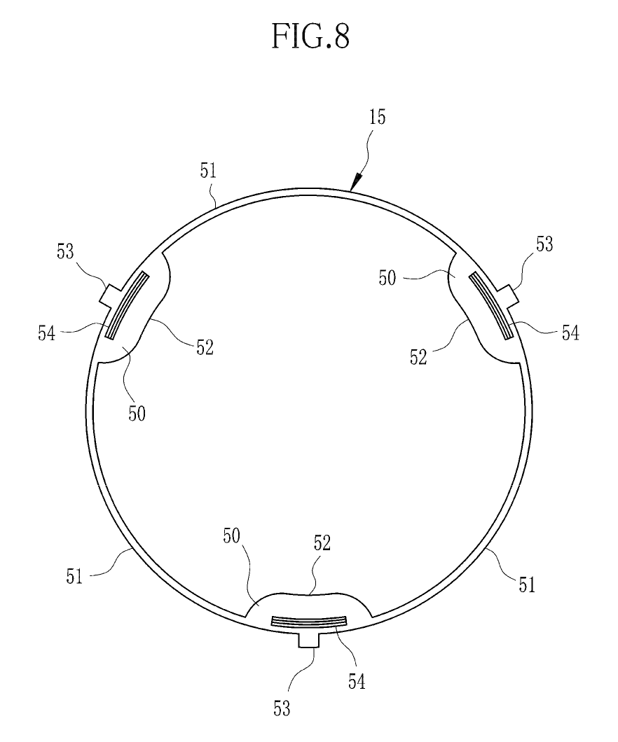

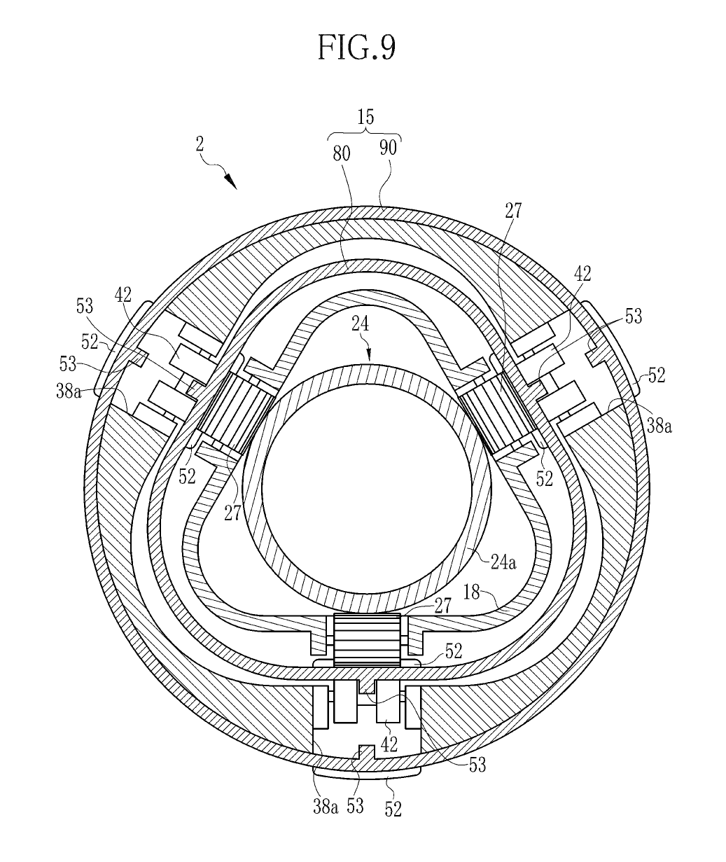

In FIGS. 5-10, the initially prepared sleeve material for forming the endless track device 15 is viewed in a cross section. The endless track device 15 is constituted by a multi-layer sheet of polyurethane resin or the like with plural film layers. Three reinforcing ridges 50 are formed on an inner sleeve surface of the endless track device 15, arranged equiangularly from one another, and formed in a trapezoidal shape as viewed in section. The reinforcing ridges 50 have a larger thickness than a membrane wall 51, and are constituted by a sheet of plural film layers of a higher number than those in the membrane wall 51. The reinforcing ridges 50 extend longitudinally in the axial direction. Engaging teeth 52 or rack gear teeth are disposed on the surface of the reinforcing ridges 50, and arranged with an inclination for mesh with the drive gears 27. Each of the engaging teeth 52 has a peripheral surface in a semi-circular shape as viewed in a cross section for contact with a wall of the body cavity where the tip device 3 advances. Also, ends of the engaging teeth 52 with respect to the transverse direction of the endless track device 15 are formed in a curved shape without a sharp form.

Guide projections 53 or ridges are formed on the endless track device 15, extend longitudinally, and are opposite to the reinforcing ridges 50. Also, a mesh sheet 54 of fiber is disposed between the engaging teeth 52 and each of the guide projections 53.

The endless track device 15 is used in the toroidal shape in FIG. 7. The three reinforcing ridges 50 are nipped between the drive gears 27 and the idler rollers 42. The drive gears 27 are meshed with the engaging teeth 52. Rotation of the drive gears 27 is transmitted directly to the endless track device 15 by the engaging teeth 52. The endless track device 15 can turn around efficiently in the axial direction. The reinforcing ridges 50 and also the mesh sheet 54 are in the multi-layer form. The engaging teeth 52 in the endless track device 15 can have sufficient mechanical strength even upon receiving driving force directly from the drive gears 27, because the engaging teeth 52 do not deform or the endless track device 15 does not break. Also, the membrane wall 51 disposed beside the reinforcing ridges 50 is effective in reducing resistance of the endless track device 15 during passage between the drive and barrel units 16 and 17.

Roller grooves are formed in respectively the idler rollers 42 at the center. The alignment ridges 53 disposed opposite to the reinforcing ridges 50 are engaged with the roller grooves when the endless track device 15 moves . Note that the barrel unit 17 can be constructed in an adjustable form for reducing the inner space of the endless track device 15 in a tightly wrapped condition. In this form, the alignment ridges 53 are engaged also with the grooves 45a and 45b of the support rings 35a and 35b. See FIG. 2. The alignment ridges 53 are effective in stabilizing the path of the movement, as the endless track device 15 can be prevented from shifting in a zigzag manner while moved in the axial direction.

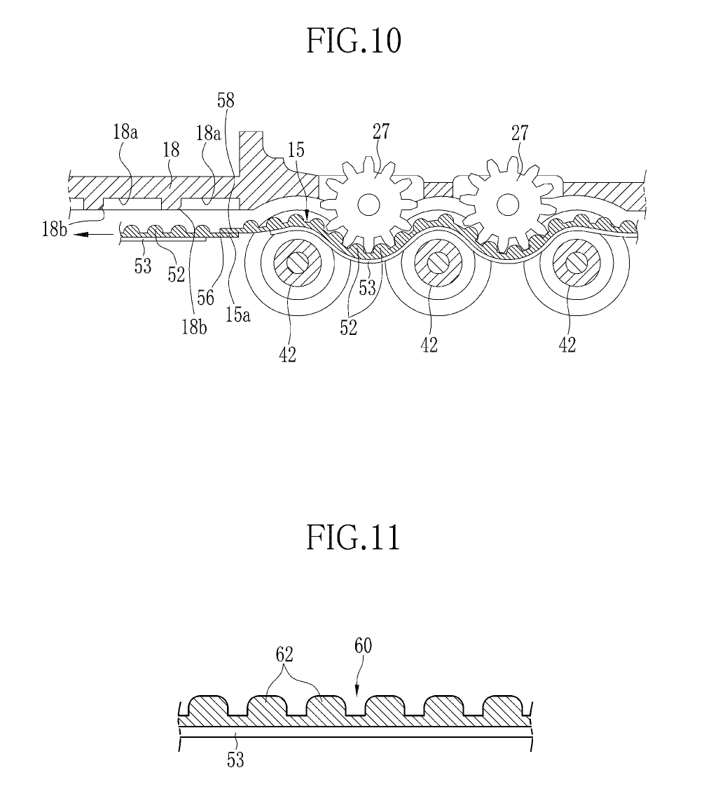

The first and second edge portions 56 and 58 of the endless track device 15 are joined together so that the second edge portion 58 on a proximal side moves in contact with the drive gears 27 in the distal direction of the arrow in FIG. 10 for the propulsion. Thus, the joint structure 15a of the endless track device 15 can be free from peeling, because drawn by rotational force of the drive gears 27.

Also, the support sleeve 18 is a part of resin formed by injection molding. Recesses 18a for saving a total volume are formed in the support sleeve 18. In FIG. 10, an inclined surface 18b is formed at a distal end of each of the recesses 18a so as to prevent interference with the second edge portion 58 at the joint structure 15a while the endless track device 15 moves endlessly. Movement of the endless track device 15 is smoothed by the inclined surface 18b.

The operation of the above embodiment is described now. In FIG. 1, the propulsion assembly 2 is mounted on the endoscope in a state of protruding a distal end of the tip device 3 partially. A special screw driving device is used for mounting the propulsion assembly 2. The clamping sleeve 20 of a clamping mechanism is rotated by the screw driving device in the clockwise direction. The clamping sleeve 20 is helically engaged with a female thread formed inside the support sleeve 18 on the distal side. Rotation of the clamping sleeve 20 in the clockwise direction shifts the clamping sleeve 20 in the inward direction or proximal direction. The tapered end surface 20a presses the sealing device 21 or C-ring. A tapered surface on a distal side of the sealing device 21 is pressed by the tapered end surface 20a to deform the sealing device 21 to decrease its diameter. The tip device 3 is squeezed by the sealing device 21 inside the support sleeve 18 upon the deformation. The propulsion assembly 2 is fastened to the tip device 3 reliably.

The wire sheath 12 extending from the proximal end of the propulsion assembly 2 is positioned along the outer surface of the steering device and the flexible device of the endoscope. Plural indicia are disposed on the wire sheath 12 equidistantly from one another, and indicate positions of attachment of the adhesive tape 4. The wire sheath 12 is attached to the steering device and the flexible device by use of the adhesive tape 4 according to the indicia. The key coupling device 13 at the proximal end of the wire sheath is plugged to the rotating coupling 14 for connection to the actuating apparatus 10, which is powered. The actuating apparatus 10 checks whether the key coupling device 13 is plugged to the rotating coupling 14 or not upon powering. If it is judged that the plugging is improper or if the plugging is not detected, alarm information is emitted, for example, alarm sound or a visible alarm signal with light. If it is judged that the plugging is proper, a sensor in the rotating coupling 14 reads type information of the propulsion assembly 2 from a signal region disposed on a bridge portion of the key coupling device 13. According to the type information, the actuating apparatus 10 automatically determines a rotational speed of the wire devices 30a and 30b and a value of a torque limiter, and prevents the wire devices 30a and 30b from operating at too high a speed or torque.

When the power source is turned on, the actuating apparatus 10 receives type information of the endoscope in connection with the processing apparatus 8 in a form of an output signal. The actuating apparatus 10 includes an inner storage medium. The actuating apparatus 10 recognizes the type information of the endoscope for use and type information of the propulsion assembly 2 by referring to table data stored in the storage medium. The table data is data of types of the endoscope and usable types of the propulsion assembly 2 in association with the endoscope types. For example, a shiftable range of the sealing device 21 is determined according to the type information of the propulsion assembly 2. An outer diameter of the tip device 3 is determined according to the type information of the endoscope. It is possible promptly to check whether the propulsion assembly 2 can be properly used in connection with the tip device 3 of the endoscope. If it is judged that a combination of the propulsion assembly 2 with the tip device 3 is improper, an alarm signal is generated, for example, alarm sound or visible alarm sign of light with an alarm lamp. Also, operation of the propulsion assembly 2 may be inhibited. Those functions can prevent occurrence of accidents.

When the foot switch 11 in connection with the actuating apparatus 10 is depressed, the motors in the actuating apparatus 10 rotate to apply torque to the wire devices 30a and 30b. The coupling gears 32a and 32b are caused to rotate, so that the spur gear teeth 24b meshed with the first coupling gear 32a are rotated with the drive sleeve 24. The second coupling gear 32b rotates in a direction opposite to that of the first coupling gear 32a. Rotation of the second coupling gear 32b is directly transmitted to the first coupling gear 32a. Thus, the motors in the actuating apparatus 10 can be utilized to rotate the drive sleeve 24.

When the worm gear teeth 24a of the drive sleeve 24 rotate, the drive gears 27, or worm wheels or roller wheels with helical gear teeth, rotate in the same direction about respectively the gear shaft 27a. The endless track device 15 is tensioned between the teeth of the drive gears 27 and the idler rollers 42 of the roller mechanisms 40. The teeth of the drive gears 27 are in mesh with the engaging teeth 52 of the endless track device 15. The idler rollers 42 are caused to rotate by the drive gears 27 to move the endless track device 15 endlessly in the axial direction of the drive sleeve 24. In FIG. 7, the drive gears 27 rotate in the clockwise direction. The idler rollers 42 rotate in the counterclockwise direction. A lower run 80 (return run) of the endless track device 15 inside the barrel unit 17 moves from the proximal side to the distal side. An upper run 90 (working run) of the endless track device 15 outside the barrel unit 17 moves from the distal side to the proximal side. Thus, the endless track device 15 endlessly turns around in the direction Y.

The upper run 90 of the endless track device 15 contacts a wall of the large intestine in entry of the endoscope with the propulsion assembly 2 in the gastrointestinal tract. While the endless track device 15 endlessly moves, propulsion force for advancing the tip device 3 is obtained, in other words, force for pressing the wall of the large intestine in the proximal direction is obtained.

The engaging teeth 52 are meshed with the drive gears 27 to transmit rotational force of the drive gears 27 to the endless track device 15. Surfaces of the engaging teeth 52 are semi-circular without a sharp form, so that a wall of the body cavity can be free from being scratched or abraded even in contact with the engaging teeth 52.

If the endless track device 15 is stopped by an increasing resistance in contact with a wall of the body cavity, the drive gears 27 are rotated with slip, because the arcuate portion of the engaging teeth 52 is slipped. Should the drive gears 27 not rotate with slip, overload occurs to a driving mechanism for the drive gears 27, namely the worm gear teeth 24a of the drive sleeve 24, the spur gear teeth 24b, the coupling gears 32a and 32b, the first and second torque wire devices 30a and 30b and the motors in the actuating apparatus 10. Those elements are likely to break. However, rotation with slip of the drive gears 27 can prevent overload to the driving mechanism. Breakage of the driving mechanism can be prevented.

Also, the endless track device 15 is formed from polyurethane resin with a resiliently deformable property. If the engaging teeth 52 are pressed by the drive gears 27 during the stop of the endless track device 15, the endless track device 15 is deformed in a direction of disengagement. Thus, the drive gears 27 can rotate reliably with slip, to prevent overload to the driving mechanism.

During the distal movement of the endoscope, foreign material stuck on the upper run 90 of the endless track device 15 may move toward the position of the lower run 80 after passing the proximal end of the barrel unit 17. However, the flange edge of the proximal cover flange 19b is positioned very close to the endless track device 15 and prevents the foreign material from internal jamming. Also, the proximal cover flange 19b prevents tissue of a body part from internal jamming together with the endless track device 15. Note that during the proximal movement of the endoscope, the flange edge of the distal cover flange 19a operates in the same manner for protection.

Light from the light source apparatus 7 is guided by the universal cable 6, a light guide device of fiber inside the endoscope, and lighting windows, and applied to the wall of the large intestine. The CCD in the tip device 3 images the wall and outputs an image signal. An output cable in the endoscope and the universal cable 6 transmit the image signal to the processing apparatus 8, to drive the display panel 9 to display an object image of the wall. A doctor or operator views the large intestine with the display panel 9.

If a lesion is discovered in the imaging, he or she enters a treatment device suitable for the lesion through an instrument channel in the endoscope. The treatment device protrudes from a distal instrument opening (not shown), and treats the lesion.

If the operator wishes to remove the propulsion assembly 2 from the tip device 3, the clamping sleeve 20 is rotated in the counterclockwise direction by use of the screw driving device. The clamping sleeve 20 shifts in an outward direction by rotating, and releases the sealing device 21 from being pressed. The sealing device 21 is enlarged by its resiliency to separate its inner surface from an outer surface of the tip device 3. The propulsion assembly 2 can be removed from the endoscope easily.

In the above embodiments, the endless track device 15 has the engaging teeth 52 of the semi-circular shape as viewed in a cross section. However, other shapes of the engaging teeth 52 without a sharp corner can be used. In FIG. 11, another preferred endless track device 60 is illustrated. Engaging teeth 62 or rack gear teeth are formed on an outer surface of the endless track device 60. As viewed in a cross section, a peripheral surface of the engaging teeth 62 has a quadrilateral shape with two curved surfaces at the corners.

In the above embodiments, the endless track device 15 is formed from flexible material. If the endless track device 15 stops, the engaging teeth 52 are disengaged by deformation to turn around with slip. However, the endless track device 15 can be rigid. An element with the endless track device 15 can be formed from flexible material to obtain the same effect. For example, the idler rollers 42 can be formed from resiliently deformable material, such as rubber. If the endless track device 15 stops, the idler rollers 42 can deform to shift the endless track device 15 away from the drive gears 27 to disengage the engaging teeth 52. Furthermore, a supporting mechanism for a rotational shaft of the idler rollers 42 can have a spring or the like for biasing toward the drive gears 27. If the endless track device 15 stops, the supporting mechanism can operate to allow the endless track device 15 to shift away from the drive gears 27.

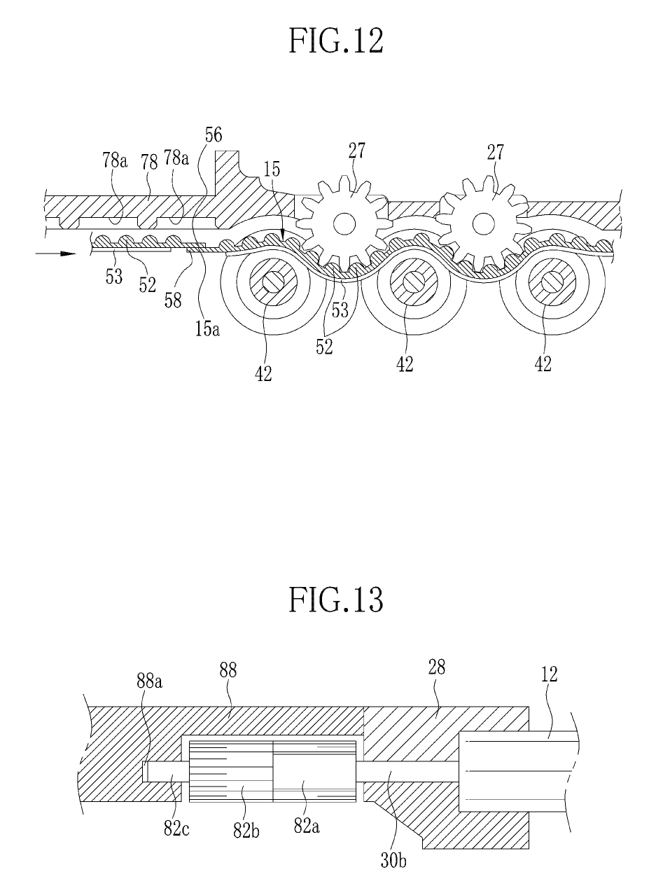

In the above embodiments, the first and second edge portions 56 and 58 of the endless track device 15 are joined together so that the second edge portion 58 on the proximal side comes in contact with the drive gears 27 for the propulsion (with the arrow in FIG. 10) . However, it is possible as illustrated in FIG. 12 to join the first and second edge portions 56 and 58 of the endless track device 15 together so that the first edge portion 56 on a distal side moves in contact with the drive gears 27 in the proximal direction with the arrow in FIG. 12. Recesses 78a for saving a total volume are formed in a support sleeve 78. In FIG. 10, an inclined surface is formed at ends of each of the recesses 78a so as to prevent interference with the first edge portion 56 at the joint structure 15a while the endless track device 15 moves in the proximal direction.

In the above embodiments, the recesses 18a and 78a are disposed on a distal side from the drive gears 27. However, the recesses 18a and 78a may be formed in the support sleeve 18 or 78 on a proximal side from the drive gears 27.

In the above embodiments, each of the coupling gears 32a and 32b is supported only on one side or in a cantilever form. In FIG. 13, another preferred support sleeve 88 with first and second coupling gears 82a and 82b or pinions is illustrated. Shafts 82c are formed to protrude from the coupling gears 82a and 82b. A support recess 88a is formed in the support sleeve 88 and supports the shafts 82c in a rotatable manner, so that each of the coupling gears 82a and 82b is reliably kept rotatable by the dual supports.

In the above embodiment, the propulsion assembly moves the tip device 3 of the endoscope forwards and backwards. However, a propulsion assembly of the invention can be a type for moving the tip device 3 of the endoscope at least forwards or in the distal direction.

In the above embodiments, the endless track device is in a toroidal shape. However, an endless track device of the invention may include a plurality of endless belts arranged in a circumferential direction of the barrel unit and extending in the axial direction.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly for mounting on a tip device of an endoscope, comprising:
a barrel sleeve having inner and outer surfaces;
an endless track device, having flexibility, disposed to extend around said inner and outer surfaces of said barrel sleeve, for endlessly moving in an axial direction of said tip device, for propulsion by contacting a wall of a body cavity;
a drive gear, engaged with said endless track device, for moving said endless track device;
a drive sleeve for rotating said drive gear;
engaging teeth, formed on said endless track device at a predetermined pitch, arranged serially, moved by said drive gear in mesh therewith, wherein a peripheral surface of said engaging teeth is curved without a sharp form.

2. A propulsion assembly as defined in claim 1, wherein said peripheral surface of said engaging teeth is arcuate according to a semi-circular shape.

3. A propulsion assembly as defined in claim 1, wherein said peripheral surface of said engaging teeth is in a quadrilateral shape as viewed in a section, and corners of said quadrilateral shape are curved arcuately.

4. A propulsion assembly as defined in claim 1, wherein said drive gear is helical and has gear teeth inclined with respect to said axial direction;
said engaging teeth are inclined with respect to said axial direction and meshed with said gear teeth.

5. A propulsion assembly as defined in claim 1, further comprising a support sleeve, disposed between said drive sleeve and said barrel sleeve, for supporting said drive gear in a rotatable manner.

6. A propulsion assembly as defined in claim 5, wherein said endless track device has an annular surface, and covers said barrel sleeve in a bag shape.

7. A propulsion assembly as defined in claim 5, wherein said endless track device includes a plurality of endless belts disposed to extend in said axial direction and arranged in a circumferential direction of said barrel sleeve.

8. A propulsion assembly as defined in claim 5, wherein said endless track device includes:
a lower run disposed to extend along said inner surface of said barrel sleeve, wherein said engaging teeth on said lower run are meshed with said drive gear;
an upper run, disposed to extend along said outer surface of said barrel sleeve, for moving in a direction reverse to said lower run in contact with said wall of said body cavity.

9. A propulsion assembly as defined in claim 8, further comprising:
at least one recess formed in an outer surface of said support sleeve;
an inclined surface, formed at least at an end of said recess on a distal side with respect to said axial direction, for smoothing movement of said lower run relative to said barrel sleeve.

10. A propulsion assembly as defined in claim 8, further comprising plural rollers, supported on said barrel sleeve in a rotatable manner, for keeping said lower run movable in cooperation with said drive gear.
